(19) United States
(12) Patent Application Publication
(10) Pub. No.: US 2026/0066108 A1
(43) Pub. Date: Mar. 5, 2026

(54) TAILOR-MADE REMOTE CELL ACTIVATION SYSTEM APP AND APPLICATION

(71) Applicants: CHUI PING HU, Taichung City (TW); Robert David Suhr, Tarrytown, NY (US); Chen Ying Chiang, Banqiao (TW)

(72) Inventors: CHUI PING HU, Taichung City (TW); Robert David Suhr, Tarrytown, NY (US); Chen Ying Chiang, Banqiao (TW)

(21) Appl. No.: 18/823,334

(22) Filed: Sep. 3, 2024

(51) Int. Cl. G16H 40/67; G06T 7/00; H04M 1/72439

(52) U.S. Cl. CPC G16H 40/67; G06T 7/0012; H04M 1/72439; G06T 2207/30024

(57) ABSTRACT

This invention uses digital electronics to mimic biology and relates to the biomedical field. It is an APP or Application installed on a computer or mobile phone which utilizes a remote cell activation system to combat ilineeses. The primary benefits include saving of resources and environmental protection, amongst other advantages.

### PRIOR ART

### Technical Field

This invention relates to the biomedical field and is an APP or Application installed in a mobile phone or computer and correlated with cell activation.

### Previous Technology

In the existing technology, cell activation mostly relies on physical contact or chemical stimulation, but these methods usually require the presence of the patient and possess certain risks. Based on the quantum entanglement theory, this invention proposes a remote cell activation system to break through the limitations of the prior art.

In the prior art, all message transmissions, in addition to needing a transmitter, also need a receiver; if the message is to be transmitted to the human body or animals or plants, the receiver must be connected or worn by the human body or animals or plants. This invention breaks through this technical concept, and does not require the target object to wear or be connected with any device for receiving information.

In the prior art, consumables for cell activation, such as drugs, can only be used once and must be remanufactured continuously, which is not environmentally friendly and expensive. The activation cell carrier made by this invention has the following advantages: permanent reuse, saving of resources and environmental protection.

### BACKGROUND OF THE INVENTION

It is known that when doctors prescribe medications for diseases such as asthma and diabetes to patients, approximately 30-40% of them do not achieve the expected therapeutic effect;
for conditions such as arthritis and Alzheimer's disease, the proportion of patients who do not benefit from the treatment is even higher, reaching 50-75%. This issue arises because traditionally if a certain medication demonstrates efficacy in clinical trials for the majority of individuals with similar symptoms, it is approved for treating patients exhibiting those symptoms, while those patients for whom the medication was ineffective in the trials are disregarded. The development of drugs using traditional systems targets the general population; however, in reality, each of us and the diseases we suffer from are unique, and many medications not only fail to be effective for some target patients but may also cause serious adverse reactions. As we gain a deeper understanding of genetic differences between individuals, personalized medicine design will become the best approach to treatment.

The above data comes from: BBC Knowledge, Issue 80, April 2018. Tailor-made precision medicine by TOM IRELAND.

Environmental pollution in the drug manufacturing process involves multiple aspects, including raw material acquisition, plant cultivation, animal extraction, chemical synthesis, pharmaceutical packaging, and transportation. The global pharmaceutical industry's carbon emissions indicate a significant environmental impact. According to research, the carbon emission intensity of the pharmaceutical industry is about 55% higher than that of the automotive industry, despite its market size being smaller and accounting for only 28% globally.

The above data comes from: How can the pharmaceutical sector reduce its carbon footprint? by ALF GOEBEL, CEO of Advanco, 25 Aug. 2021.

The following details the main environmental pollution scenarios in each stage:

### Raw Material Acquisition

1. Chemical Synthesis: The pharmaceutical process uses a large amount of chemicals which are released into the environment during production involving the air and bodies of water. These chemicals may include organic solvents, heavy metals, and other toxic substances.
2. Plant Cultivation: Plants used in pharmaceuticals often require large amounts of pesticides and fertilizers, which can leach into the soil and bodies of water, causing agricultural pollution.

### Manufacturing Process:

1. Chemical Synthesis and Pharmaceutical Processes: These processes require a large amount of energy, particularly electricity and heat, which primarily come from fossil fuels, resulting in high carbon emissions.
2. API Production: The production of active pharmaceutical ingredients (APIs) releases significant pollutants, further increasing carbon emissions.

### Supply Chain and Transportation:

The global pharmaceutical supply chain, which involves transportation to production sites from locations worldwide, involves burning fossil fuels, resulting in significant carbon emissions. Reports indicate that supply chain emissions account for over 80% of total pharmaceutical emissions.

The above data comes from: Global Chemicals Outlook II, UNEP, 2019.

### Waste Treatment:

The treatment of wastewater and solid waste generated during the pharmaceutical process also consumes substantial energy, further increasing carbon emissions.

### Solution

1. This invention proposes an innovative remote cell activation system that utilizes quantum entanglement for remote information transmission. By integrating a dynamic carrier database, the system can provide customized cell activation solutions based on user needs. This invention not only reduces the need for long distance transportation but also helps to effectively lower overall carbon emissions, offering extensive application prospects and significant market potential.

Especially after the outbreak of the Covid-19 pandemic, the medical technology industry should further research the production of remote digital electronic drugs and the technology of remote transmission of digital electronic drugs to the target, as well as the development of digital electronic vaccines.

If the human living environment continues to deteriorate, physical drug shortages will make it difficult to cope with potential pandemics that may occur every ten years. It is hoped that the biotechnology and medical fields will continue to promote research on more effective remote cell activation system technologies, which can reduce contact between doctors and patients, breakthrough transportation's plight, and instantly transmit cell activation carrier information corresponding to pandemic strains for global users to download and use.

2. This invention is to choose to use computers and mobile phones as a device:

Technological innovation should make human life better, not produce a worsening climate warming crisis.

In 2009, a team of scientists, led by Johan Rockström, proposed the concept of planetary boundaries. The concept was first published in 2009 and updated in 2015.

Our priority is to halve global carbon emissions by 2030 and achieve net-zero emissions by 2050. This means that all systems related to life must be zero carbon emissions.

However, we found that every time we bought an electrical device, there were different wires and ancillary equipment, and the next time the product was updated, there were more new wires and equipment. People always have a bunch of cables at home and a bunch of obsolete devices. Those technological products' waste is one of the culprits of environmental damage.

Therefore, this invention makes use of the almost necessary equipment for today's modern people, that is, computers and mobile phones, which can surf the Internet, record audio, take pictures, execute APP, and update the characteristics of the application system to invent a way which lets devices generate three different electric fields to develop the remote cell activation system; and using the entanglement effect to create a way which uses the technology of wireless and infinite transmission to send the final converted electric field information to the target object.

This design, which uses an App and application as command centers for issuing instructions, and computers and mobile phones as execution devices, can seamlessly integrate AI medical diagnosis systems and AI robots to provide more accurate mobile medical services for humans and other organisms.

### SUMMARY OF THE INVENTION

### Advantages

The invention is to provide a remote cell activation system that utilizes digital electronic technology to bionically target the object to obtain correlated quantum entanglement pairs' information. This invention orders the devices' camera takes pictures or the recording device records the voice, and then uses either one to obtain the quantum entanglement pair's digital data from the target object. Users can use appropriate cell activation carriers that has been established in the program of this invention according to the type of the target object different types of cell activation needs. Users can also use the invention to establish a new activation cell carrier according to the needs of target objects for different field. Finally, through a computer or mobile phone to carry out the cell activation operation. Although the invention is installed in a mobile phone or a computer, the principle of entanglement effect is applied; there is no need to contact the target object when carrying out the operation, no Internet connection is required and there is no distance limit. Moreover, all cell activation carriers can be reused permanently.

### BRIEF DESCRIPTION OF DRAWINGS

FIG. 1 shows the architectural design of the remote cell activation system of this invention.

FIG. 2 shows a flow chart of quantum entanglement pair digital data extraction of this invention.

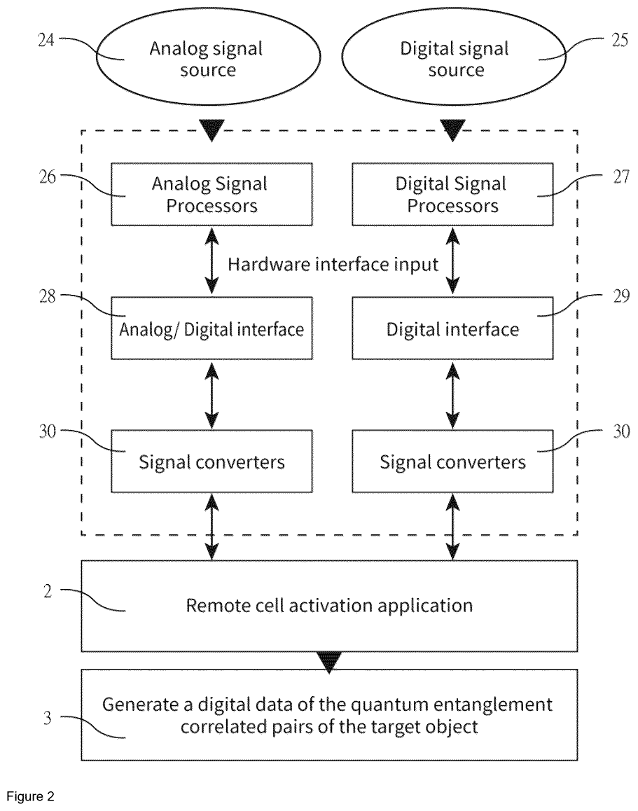

FIG. 3 shows a flow chart for the selection and application of cell activation carriers of this invention.

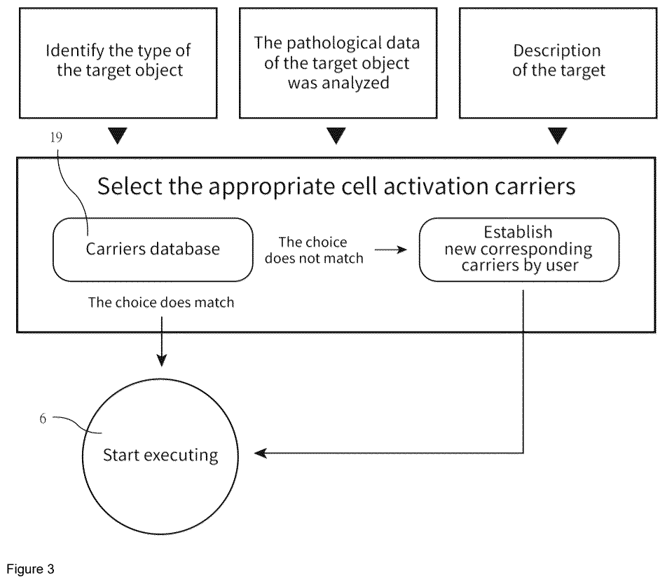

FIG. 4 shows a schematic diagram of the execution interface of the system of this invention's computer version.

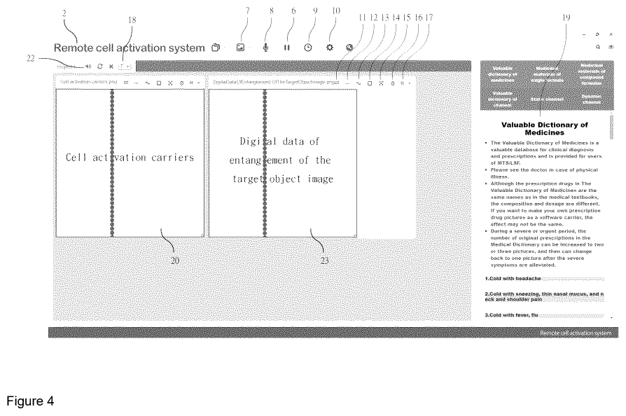

FIG. 5 shows a schematic diagram of the execution interface of the system of this invention's mobile phone version.

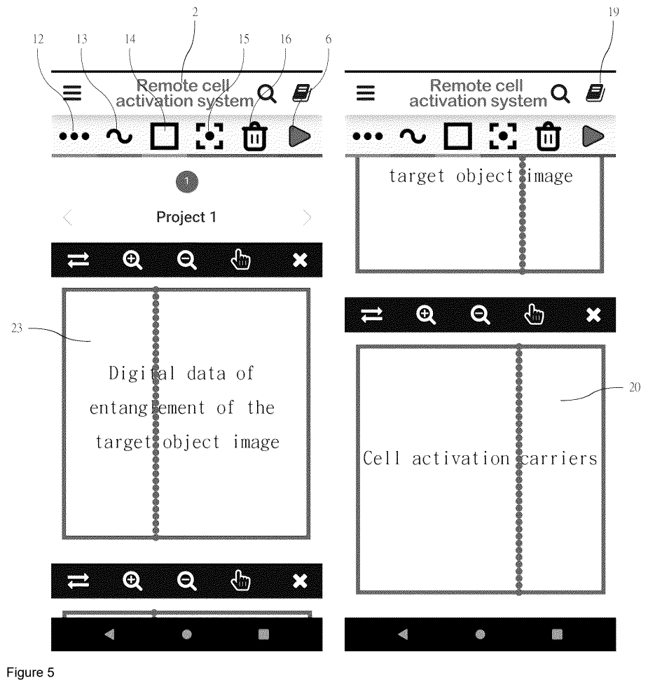

FIG. 6 shows the activity of the plasma when the computer screen develops photos.

FIG. 7 shows the electric field state when the system of this invention executed.

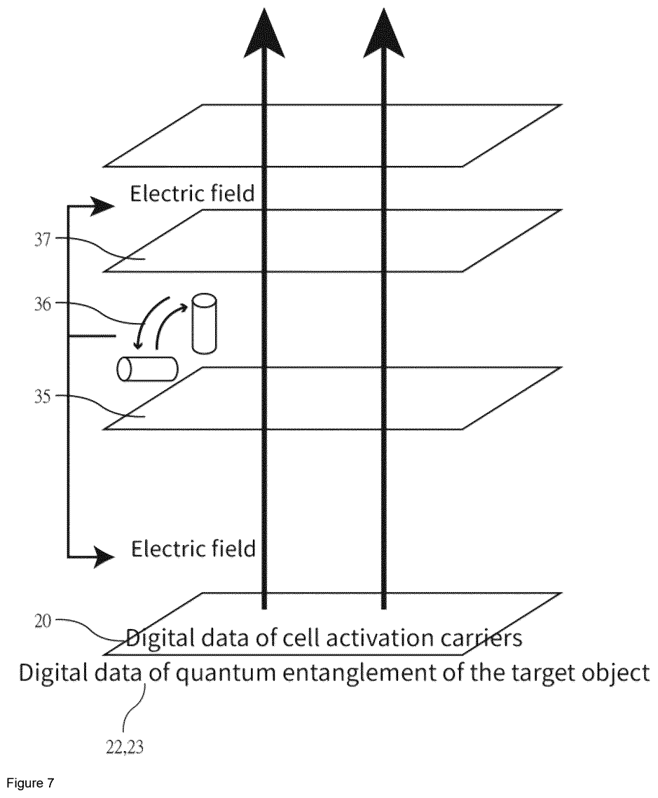

FIG. 8 shows the system APP and application plane execution diagram.

FIG. 9 shows the system APP and application side execution diagram.

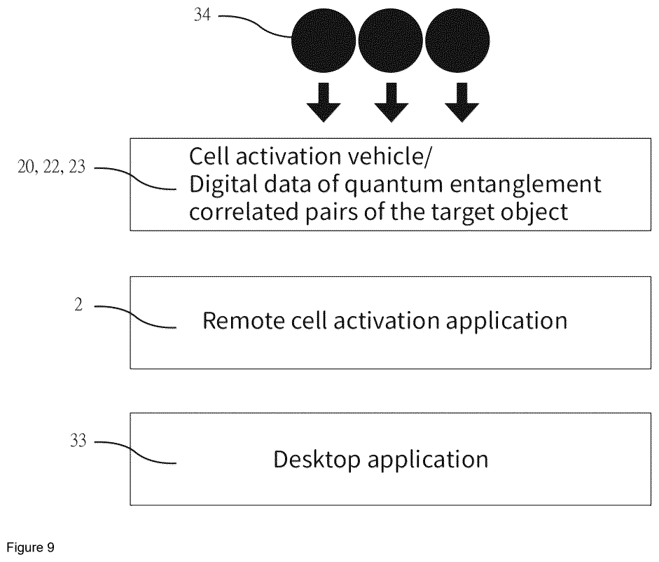

FIG. 10 shows the system of computer version of this invention about the digital image of the cell activation carriers and the execution state to target the cell area which require activation.

FIG. 11 shows the current change of the electric field when the system of this invention executes the dynamic meridian system.

FIG. 12 shows the computer version system of the invention. The cell activation digital image carrier consists of many layers upon execution.

FIG. 13 shows the Mother electric field and Child electric field are transmitted through entanglement pairs.

FIG. 14 shows that when computers and mobile phones are turned on, electromagnetic field A is formed.

FIG. 15 shows that when the APP and application of the cell activation system are activated electromagnetic field B is formed.

FIG. 16 shows that when the sound or image of the target object is continuously activated an electromagnetic field C is formed.

FIG. 17 shows a schematic diagram of the execution of the APP's mobile phone version.

### DESCRIPTION OF THE NUMBER ON DRAWING

1: Remote cell activation application system architecture
2: Remote cell activation application
3: The produced unit of quantum entanglement correlated pairs of the target object
4: Frequency-modulated unit of quantum entanglement correlated pairs
5: Quantum entanglement correlated pair execution unit
6: Quantum entanglement correlated pair transmit unit
7, 8, 9, 10, 11, 12, 13, 14, 15, 16, 17, 18: Frequency-modulated unit
19: Digital database of cell activation carriers
20: Digital data of cell activation carriers
21: Target object
22, 23: Digital data of quantum entanglement pairs of the target object
24: Analog signal source
25: Digital signal source
26: Analog signal processors
27: Digital signal processors
28: Analog/Digital interface
29: Digital interface
30: Signal converters
31: computer
32: cell phone
33: operating system
34: Digital red spots
35: Front-conductive glass
36: Liquid crystal electronic activity area
37: Thin-Film Transistor
38: Atoms activity irregular
39: Atoms activity regular

### DETAILED DESCRIPTION OF THE INVENTION

### Implementation Methods

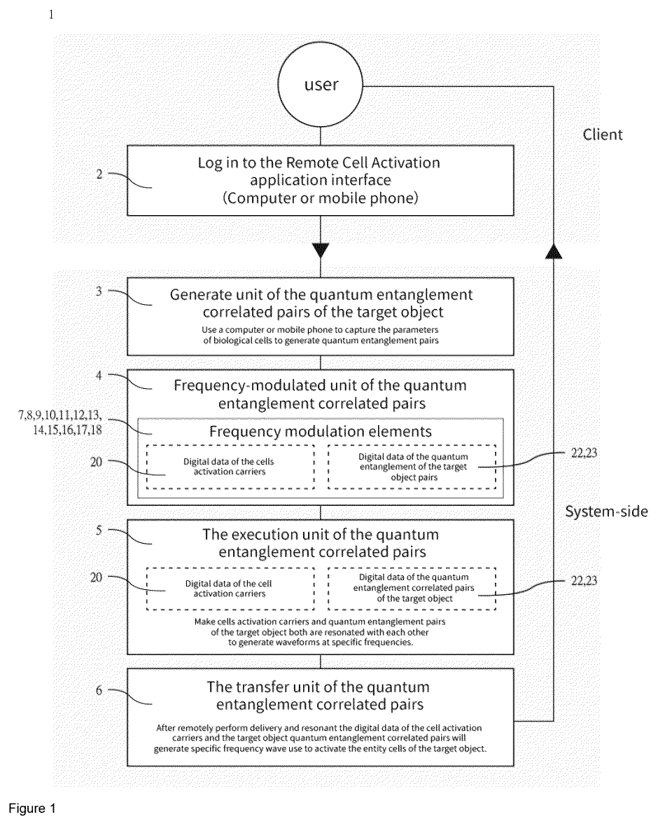

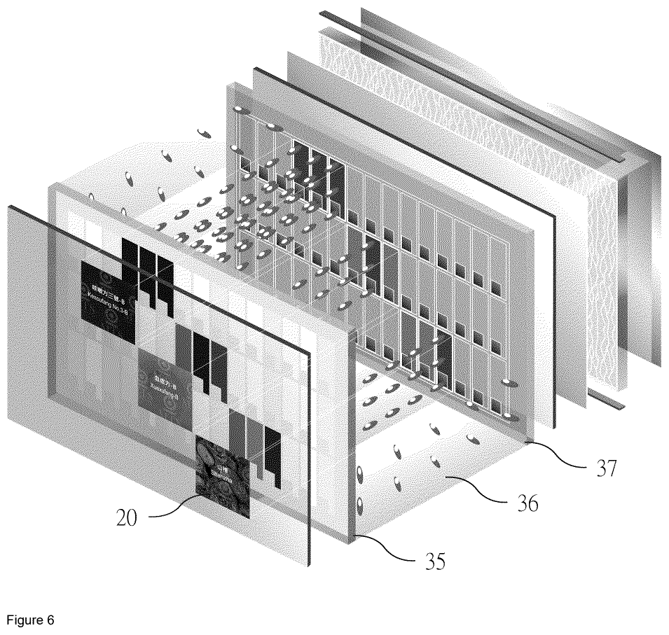

The implementation methods of the present invention are divided into two parts:
1. Software technology: APP and Application FIG. 1,2,3,4,5,8,9,10,12,17
2. Hardware technology: about mobile phones and computers FIG. 6,7,11,13,14,15,16,17

### Part I: Software Technology: Apps and Application

Extract the digital data of the target object entanglement pair. There are two ways to do this:
Method 1: Capture images: The user uses the camera of a computer or mobile phone to shoot the image data of the target object. The image data is converted into digital data to obtain a fluctuation frequency which is similar to the target object.
Method 2: Record sound: The user uses the microphone of the computer or mobile phone to record the sound data of the target object. Convert sound data into digital data to obtain a fluctuation frequency which is similar to the target object.

Select the cell activation carriers as explained below:
Depending on the type of organism's need, to add the carriers suitable for this type of cell to activate.
   Judgment method:
(a) According to the identity of the type of the target object.
(b) According to the pathological analysis report of the target object.
(c) According to the description of the target object.

That is to say, a user wants to improve a skin cell area which got wounded, he/she can select and add the corresponding cell activation carriers, for example, a digital imaging that can promote regenerative capacity.

In order to allow users flexibility and fully utilize the resources of this system, this invention was specially designed and built with the following functions:
(1) The invention has a designed carrier database that can be dynamically used to create additional data according to different cell activation needs.
(2) The invention allows users to select the corresponding cell activation carrier from the preset carrier's database according to the user's needs and the type of cell activation.
(3) If there is no applicable carrier in the database, the invention has the function that provides the user to establish a new corresponding carrier by themselves.

### Software Execution Techniques:

1. Remote control: Through quantum entanglement pair technology, the computer or mobile phone ensures that the information of the cell activation carrier can be accurately transmitted to the target object.
2. Cell activation: Perform cell activation operations through computers or mobile phones, and use quantum entanglement pair technology to continuously transmit activation information to target object to achieve activation effects, adjustments and optimizations.

### Part 2: Hardware Technology: Mobile Phones and Computers.

### Hardware Execution Techniques:

The invention utilizes the background design of the APP and application to command mobile phones and computers to perform work to generate the cell activation, as follows:

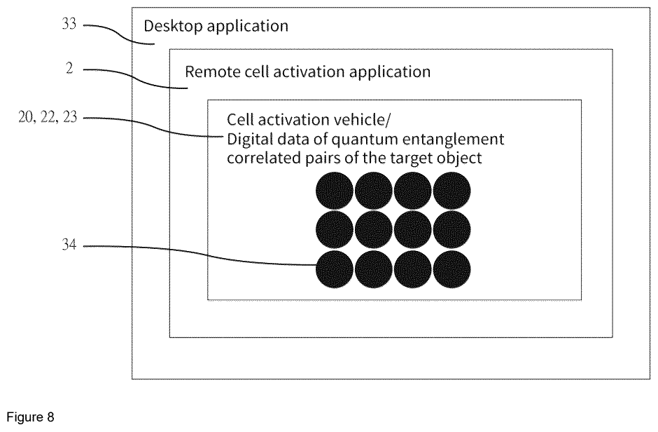

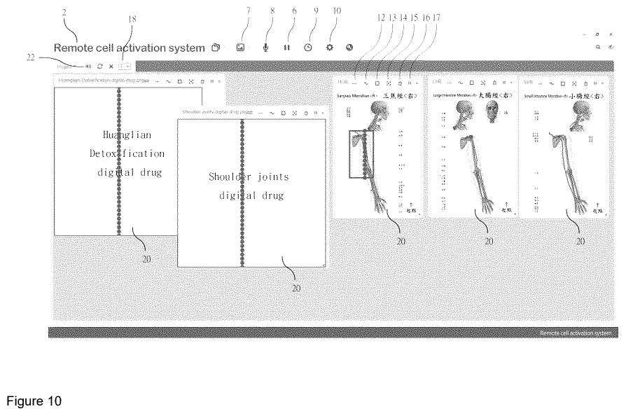

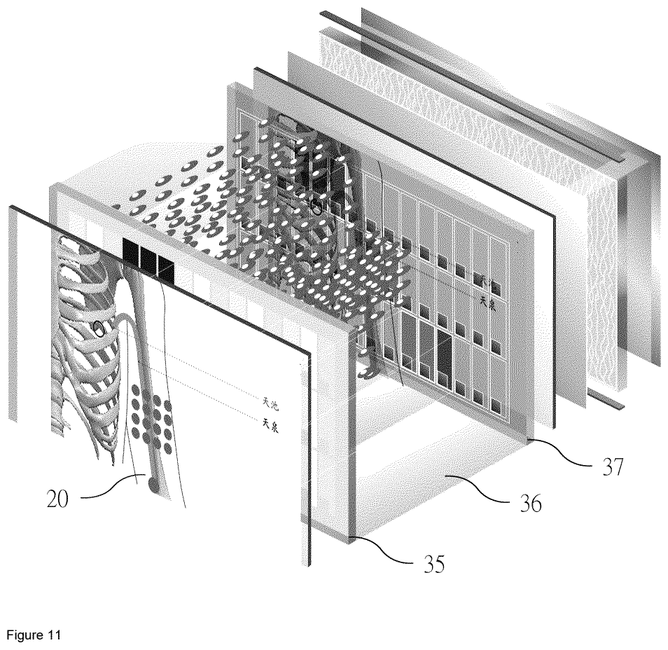

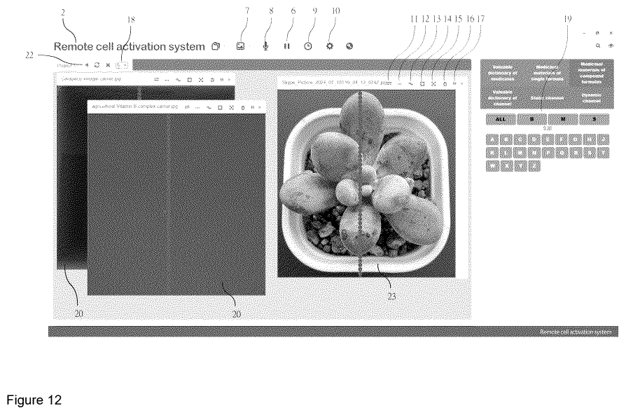

It is a fact that when the computer is powered on, we can detect electromagnetic waves, which means that the entire machine is an electric field, and its electronic activity is expanded. Therefore, the invention exhibits the following control and design:
(a) As shown in FIG. 6, 7, there is frequent electronic activity between the display and the driver of the hardware, and this electronic activity forms an electromagnetic field. This invention has a program designed to perform a scanning and clicking action, and fully utilizes this electromagnetic field, as shown in FIG. 8, 9. The invention's program can execute a flashing red dot, so that the red dot will provide a pulsating action on a photograph. The naked eye sees the red dot clicking, but it is actually using the pixel to flash, that is, it operates through the superposition of high voltage, low voltage, and electrons. In order to form a stable electric field and continuously transmit resonant waves during execution, the action of clicking on the carrier must be continuous.
(b) The image types in the invention are basically divided into two kinds, one is the quantum entanglement pair digital data of the target object, and the other is the digital data of the cell activation carrier. The purpose of having the program scan/click on an image is to enhance the natural frequency of the image. See FIG. 8, 9.
(c) The invention is designed to target a specific position in the anatomical diagram or the meridian diagram, so that the electronic activity of the specific position increases, and the bonding resonance between the electronic activity of the specific position and sound can be enhanced. For example, when the scapula and elbow joints are injured, the scapula and elbow joints can be framed, a cell activation vehicle can be added, and then a red dot click scan can be performed to strengthen the natural frequencies of the area and help activate the cells in the area. See FIG. 10
(d) For example, see FIG. 11. This invention expands the design of the dynamic meridian system and increases the current activity to promote the activation of cells in the whole region.
(e) For example, see FIG. 12. This invention can select a variety of cell-activated digital carriers at the same time according to the requirements of the target object through the phenomenon of both an electron tunneling effect and electron sharing, and when the number of cell-activated digital image carriers is large enough to occupy the entire screen, the second layer image is covered by the first layer of images, and after that, the third, the fourth, and the eighth layers are all effective. In particular, the screen of a mobile phone is small, and as we need to use more than two carriers, layers of overlap will inevitably occur. When the application starts executing, all subroutines that belong to this app will continue to calculate and react even behind the scenes.

Deliver the cell activation carriers to the target object. There are two ways to do this:
(a) Through digital data of the entanglement pairs of the sound that comes from the target object.
(b) Through digital data of the entanglement pairs of the image that comes from the target object.

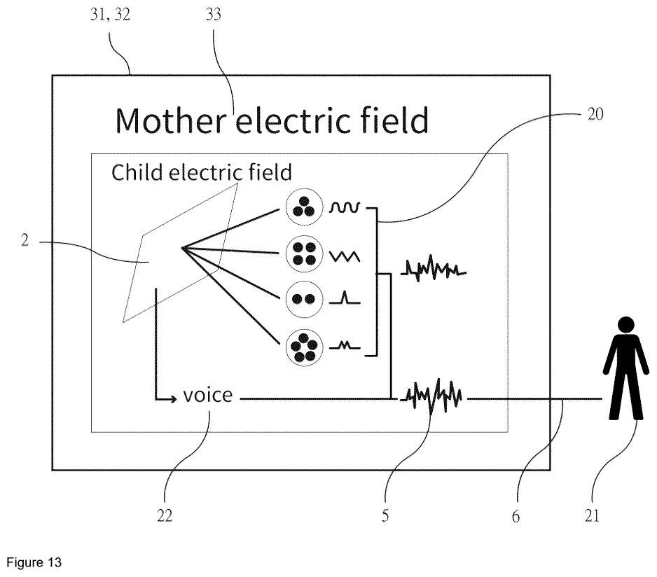

Utilized the technology of the quantum entanglement pairs explained below:
(a) The invention must continuously play the sound of the target object or continuously click and scan the image of the target object as a carrier wave through the phenomenon of the entanglement effect, and transmit the fluctuations of the computer or mobile phone to the target object when performing resonance.
(b) When playing the sound continuously, the sound does not need to be turned on loud, it can be turned to the lowest sound, but not to mute, and you can also use headphones. The target object does not need to hear the sound, nor need to see the screen image executed by the computer or mobile phone.
(c) There is electronic activity on the digital picture and there is electronic activity on the photo on the monitor. The red dot click is not really clicking on the photo, they are two different system circuits, but the electrons developed in the photo have been moving, and the red dot has been moving, and finally an electric field will be formed; this will produce a magnetic field, which will spread in all directions, but there is only one magnetic field that fluctuates with the same sound or the same image. So, the mechanism formed, as shown in FIG. 13, constitutes the mother field in the mobile phone or computer. A sub-field is an App or an application program, and the circles represent different digital photos, which have their own fluctuations. This invention gives meaning to this wave to add the sound or image of the target object, which belongs to the same quantum entanglement pairs with the real target object, and the fluctuation frequency which is similar, and the entangled resonance phenomenon can be produced.
(d) The digital sound of the target object is continuously played in the mother field or the digital image of the target object is continuously scanned with the red dot, and the converted fluctuations can be transmitted to the target object no matter how far apart they are.

The target object does not need to wear or connect to a device to receive information from the cell activation carriers. The execution technology is explained below:
(a) The target object described in the invention has an electromagnetic reaction, can send an electronic signal, and can also receive electronic signals. For example, the human body is a conductor of electricity. It can be compared to an antenna, but the structure of the antenna is different and the composition elements are also different, but electronic activity in the human body and antennas are the same. Ultrasound, X-ray development, tomography, MRI, etc., all diagnose diseases by interpreting the electronic activity in the human body.
(b) To be specific: the human body is a veritable electronic transceiver that can send and receive electronic signals. The human body is an antenna when it receives information, and the human body is a radio station when it sends information. This invention relies on the sound or image of the target object, which is also the entanglements' pairs, to lock on to the target object and transmit an electronic signal by means of the entanglement effect, and the human body or animal or plant that is the target object can receive the message without wearing a receiver.

This invention has enabled the use of computers and mobile phones to manufacture cell activation carriers and has developed the electromagnetic field technology of resonant cell activation carriers.

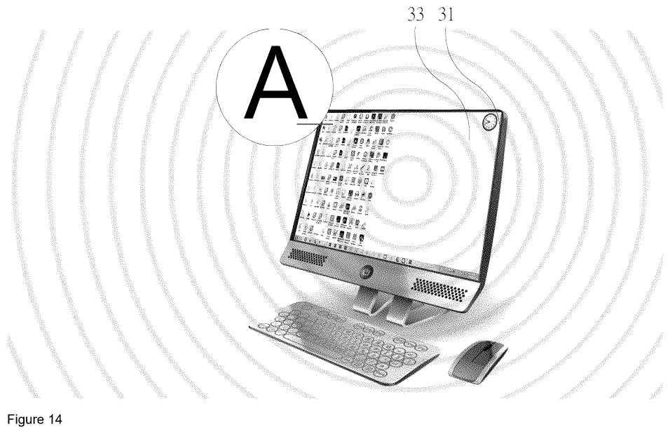

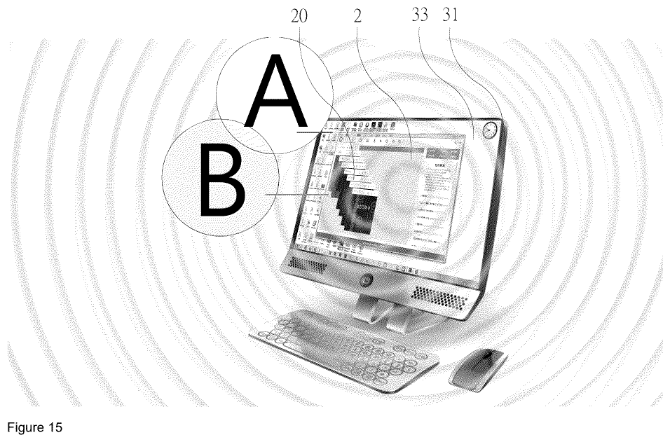

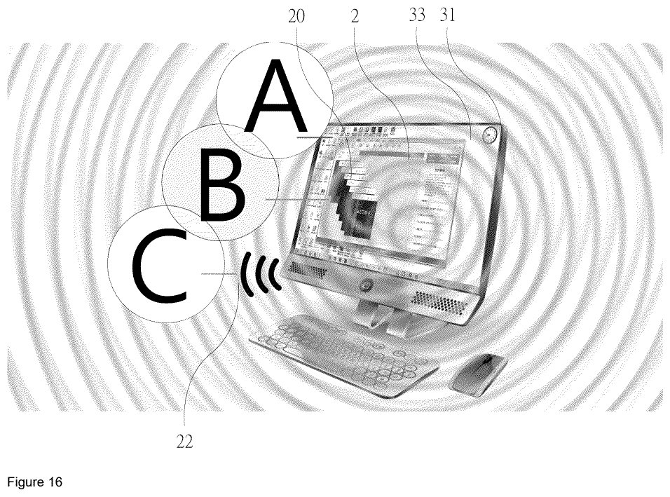

About the technology that produces three electromagnetic fields, explained below:
(a) When the computer or mobile phone is turned on, the device will form an electromagnetic field A. See FIG. 14. In A, we will separately manufacture the electromagnetic fields B and C.
(b) As the APP and the applications program of cell activation systems begin to operate, electromagnetic field B is formed. See FIG. 15.
(c) When the sound of the target object is played continuously, an electromagnetic field C is formed. See FIG. 16.
(d) Let A, B and C electromagnetic fields operate at the same time. When A, B and C electromagnetic fields operate at the same time, they will begin to form an overlapping resonance. This kind of overlapping resonance activity is actually an electronic activity, because the digital coding units are the same. See FIG. 16.

The way to transmit the overlapping resonant message to the target object:

The main way to transmit information to the target object is to play the sound or scanned photo of the target object utilizing a computer or mobile phone.

In the technical aspect of this invention, the main functions of the digital sound and image of the target object are:

Lock on to the target object that is emitting the sound. This is because the sound emitted by the target object and the sound played from the computer or mobile phone originate from the same target object. As a result, even if the computer or mobile phone playing the sound is far away from the target object, they can also experience entanglement resonance.

Sound and image of target objects are carrier waves which transmit the fluctuation of the cell activation carriers.

All carriers, sounds and images are converted by Fourier in the electric field inside the computer or mobile phone, and each set of electronic activities after conversion contains sound fluctuations. We can compare the wave of sound to a conveyor belt, transporting these packets to the target object that emits the sound.

Ways to continuously intensify the entanglement effect of entanglement pairs, explained below:
(a) During the execution of the resonance period, the computers and mobile phones must play sound or scan the image of the target object constantly; the sound does not have to be turned up, it can be turned to the minimum setting, but cannot be muted.
(b) The target object does not need to hear the sound, nor does he/she/it need to see an image from a computer or mobile phone when it is executed.

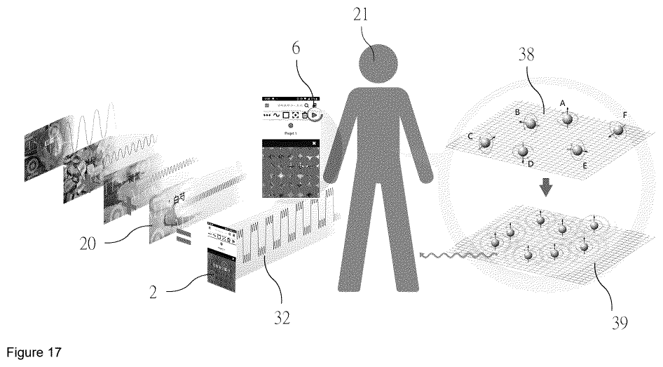

The transformation of advantages over the target object after performing resonance and transmission:
(a) When the human body, animals and plants are sick, the state of atomic activity is chaotic, and the cells are weakened. See FIG. 17. Therefore, perform cell activation operations to assist the body in repairing.
(b) Since the fluctuation of the carrier output by the computer or mobile phone is stable; when the fluctuating particles of these carriers hit the particles inside the target, it resonates with the particles inside the target object, causing a harmonic oscillation effect. This effect can gradually adjust the irregular fluctuations formed from the target object due to disease to become stable and orderly fluctuations, so as to achieve the purpose of cell activation. Such as FIG. 17.
(c) When the human body, animals and plants get sick, their functions will be in a dull or weakened state. It may not have the ability to activate the immune response to kill bacteria. The Remote Cell Activation System can assist in cell activation, improving the body's ability to fight germs; the operator can select or add suitable carriers according to the different discomfort symptoms and types of the organism to achieve the purpose of cell activation.

### EMBODIMENT EXAMPLES

### Operating Procedures:

1. Use the computer's camera to take images or the recording function to record sound and obtain the digital data of quantum entanglement pairs through the application. Select carriers suitable for immune cell activation, and remotely transmit it to the target object for remote cell activation.
2. Use the mobile phone camera to take images or the recording function to record the sound. Then obtain the digital data of the quantum entanglement pair of the target object through the APP, select the carrier suitable for tissue cell repair, and remotely transmit it to the target object for remote cell repair operation.

(Embodiment 1): A target object needs to be activated remotely. The system takes an image of the damaged skin of the target object through a mobile phone, records the sound of the target object, selects suitable cell activation carriers, and remotely transmits it to the skin cells of the target object through quantum entanglement pair technology to realize remote cell activation.

(Embodiment 2): In an agricultural application, plant cells need to be activated remotely, transmitted to the system by taking plant images, identifying the cause of plant cell weakness according to the images, selecting suitable chemical molecule digital data as cell activation carriers, and remotely transmitting to plant cells through quantum entanglement pair technology to realize remote cell activation.

### Example 1

Plants in a horticultural center are infected with a fungus; the bacterial index of the experimental strain was 8126 before the experiment, and the index was reduced to 6911 after 5 days 6 hours per day after executing remote cell activation of the carapace vinegar digital carrier. Then, after continuing for another 5 days, the number of bacteria was 13967, and the plant was much stronger than 10 days ago. Plants infected by the fungus will gradually decay and die if not rescued. The plant has an increased number of bacteria, but it is stronger than 10 days ago, then the 15th days, the number of bacteria was 20051. That is because the good bacteria have overpowered the bad bacteria; if the plant is getting stronger, it means that the cells of the plant are activated, the good bacteria have increased, and the immunity against bad bacteria has begun. The main purpose of this experiment is to prove that when a plant is sick, using the right cell activation carriers can prevent the symptoms from worsening and the plant will gradually recover their health.

TABLE 1

| | The experimental |
|---|---|
| | plant |
| The number of bacteria measured before the | 8126 |
| experiment | |
| Carapace vinegar digital carrier resonance day 5 | 6911 |
| (6 hours per day) Measured bacterial count | |
| Carapace vinegar digital carrier resonance day 10 | 13967 |
| (6 hours per day) Measured bacterial count | |
| Carapace vinegar digital carrier resonance day 15 | 20051 |
| (6 hours per day) Measured bacterial count | |

Because the plant was only given water during the experiment, and not given nutrient fertilizer, the number of good bacteria will be reduced if they lack nutrients when they multiply. The use of agricultural vitamin B complex carrier resonance, although it does not actually give nutrients, provides fluctuations in the activation of good bacteria, observes the plant, stays strong, and grows new leaf layers.

TABLE 2

| | The experimental |
|---|---|
| | plant |
| Carapace vinegar digital carrier Resonance Day 23 | 8969 |
| (3 hours per day) | |
| Measured bacterial count | |
| Carapace vinegar digital carrier and agricultural | 11495 |
| Vitamin B complex carrier, 1:5 dilution Resonance | |
| Day 29 | |
| (3 hours per day) | |
| Measured bacterial count | |
| Carapace vinegar digital carrier and agricultural | 14159 |
| Vitamin B complex carrier, 1:5 dilution | |
| Resonance Day 38 (3 hours per day) | |
| Measured bacterial count | |

### Example 2

1. The first experiment: Pour 10 cc of fermented food waste water into each petri dish and resonate with 10 cc of bleach.

TABLE 3

| | Control | Experimental |
|---|---|---|
| | group | group |
| The number of bacteria was measured | 67 | 23 |
| before the experiment | | |
| Resonance with10 cc of bleach; the | 86 | 50 |
| bacterial count was measured after 1 hour | | |
| Resonance with 10 cc of bleach, the | 47 | 125 |
| bacterial count was measured after 2 hours | | |

2. The second experiment: Pour 10 cc of fermented food waste water into each petri dish and resonate with 600 cc of bleach.

TABLE 4

| | Control | Experimental |
|---|---|---|
| | group | group |
| The number of bacteria was measured | 3 | 3 |
| before the experiment. | | |
| Resonance with 600 cc of bleach, the | 3 | 6 |
| bacterial count was measured after 1 hour | | |
| Resonance with 600 cc of bleach, the | 9 | 22 |
| bacterial count was measured after 2 hours | | |
| Resonance 600 cc bleach, the bacterial | 4 | 13 |
| count was measured after 3 hours | | |
| Resonance 600 cc bleach, the bacterial | 13 | 12 |
| count was measured after 4 hours | | |
| Resonance 600 cc bleach, the bacterial | 4 | 10 |
| count was measured after 5 hours | | |

The results of this experiment are explained below:
(a) The safety of the remote cell activation system, even with the use of a large amount of a bleach digital carrier will not harm the organism.
(b) The number of bacteria in the experimental group was higher than that in the control group, because the experimental group was targeted by operating resonance, and the cells in the experimental group were still activated and replicated even under harsh environmental conditions, the high doses of bleach.

3. The third experiment: Pour 20 cc of fermented food waste water into each petri dish, and resonate the agricultural vitamin B complex that is effective for the reproduction of food waste bacteria, 1:5 dilution.

TABLE 5

| | Control | Experimental |
|---|---|---|
| | group | group |
| The number of bacteria was measured | 2 | 3 |
| before the experiment | | |
| Resonant B vitamins, the bacterial count | 9 | 29 |
| was measured after 1 hour | | |
| Resonant B vitamins, the bacterial count | 4 | 5 |
| was measured after 2 hours | | |
| Resonant B vitamins, the bacterial count | 4 | 5 |
| was measured after 3 hours | | |
| Resonant B vitamins, the bacterial count | 7 | 4 |
| was measured after 4 hours | | |
| Resonant B vitamins, the bacterial count | 2 | 5 |
| was measured after 5 hours | | |

The results of this experiment are explained below:
(a) Providing the appropriate activation cell carriers help to activate the cell.
(b) The number of cell activation replicates in the experimental group was higher than that in the control group.
(c) Because no nourishment is provided to the two groups of bacteria, the bacteria in the petri dish will gradually decay and die after multiplication.

1. This invention aims to provide a remote cell activation system based on quantum entanglement effect and biomimetic science that can be tailored through its application method. This invention utilizes the digital electronic technology of a mobile phone and a computer to carry out biomimetic targeting on organisms, including the extraction of entangled correlated pairs of image and sound information, the extraction of digital data of cell activation carriers, the selection and optimization after extraction, the application technology of electric fields, and the execution and transmission of remote cell activation using quantum entanglement pair technology. Users can add suitable cell activation carriers according to different types of cell activation needs, and finally send cell activation carrier messages to the target object via computer or mobile phone, which does not need to wear a message receiving device or connect to the Internet. The extracted image and sound information and digital data of the cell activation carriers can be permanently reused.

2. This invention's technique of claim 1 wherein said which executes an APP and application of THE TAILOR-MADE REMOTE CELL ACTIVATION SYSTEM through a computer or a mobile phone.

3. This invention's technique of claim 1 wherein said which uses a computer and a mobile phone to act as an electric field that emits cell activation carriers.

4. This invention's technique of claim 1 wherein said is based on the quantum entanglement effect and the phenomenon of electronic superposition sharing to remotely transmit and receive digital information. The target object has no need to wear a device or integrate with any kind of connecting line.

5. This invention's technique of claim 1 wherein said creates a way that the cell activation digital data can be reused for an unlimited number of times after being made into a carrier.

6. This technique of claim 1 wherein said involves taking images to obtain quantum entanglement correlated pairs of digital data of the target object can be reused for an unlimited number of times.

7. This technique of claim 1 wherein said that makes a living body or object into a cell-activated digital carrier that can be permanently reused.

8. This technique of claim 1 wherein said that invented the method of photographing image data to obtain quantum entanglement correlated pairs of digital data.

9. This technique of claim 1 wherein said that invented the method of recording sound data to obtain quantum entanglement correlated pairs of digital data.

10. This technique of claim 1 wherein said invented a method that uses quantum entanglement effect to remotely transmit cell activation carriers to the target objects by photographing or recording the sound of target objects.

11. This technique of claim 1 wherein said invented an applications and operation techniques for selecting and optimizing digital images of cell activation carriers.

12. This invention's technique of claim 1 wherein said protecting the right of the cell activation carriers' digital images database of the system.

13. This invention's technique of claim 1 wherein said that transmits cell activation carriers' information onto the target object which contains the similar quantum entanglement pairs.

14. This invention's technique of claim 1 wherein said produces 3 electromagnetic fields to enable the cell activation digital carriers to fluctuate.

15. This invention's technique of claim 1 wherein said after the three electric fields of A, B, and C are activated at the same time, the C electric field of the quantum entanglement pair of the target object is used as a technology that can still emit directional wave information when tracking the movement of the target object.
